(19) United States
(12) Patent Application Publication
(10) Pub. No.: US 2011/0106258 A1
(43) Pub. Date: May 5, 2011

(54) END CAP FOR A VERTEBRAL IMPLANT

(75) Inventors: Jonathan Blackwell, Arlington, TN (US); Thomas E. Drochner, Memphis, TN (US); Michael J. Merves, Memphis, TN (US)

(73) Assignee: WARSAW ORTHOPEDIC, INC., Warsaw, IN (US)

(21) Appl. No.: 12/609,229

(22) Filed: Oct. 30, 2009

(51) Int. Cl. A61F 2/44

(52) U.S. Cl. 623/17.16

(57) ABSTRACT

An implant for insertion between adjacent vertebral members, comprising an implant body with at least one base section and an end cap. The end cap is adapted for selective positioning on a base section via rotational adjustment of the end cap. The end cap comprises an exterior contact surface that faces away from the implant body when positioned on the base section, a seating surface which contacts the base section when positioned on the implant body, end cap angulation, positioning passages adapted to receive positioning teeth, the positioning passages and positioning teeth complimentarily configured to facilitate positioning the end cap on the base section, and a locking mechanism adapted to lockingly engage the end cap and base section such that the implant imparts end cap angulation to an adjacent vertebral body at the selected point when the implant is positioned in the intervertebral space.

### BACKGROUND

The present application is directed to implants, devices and methods for stabilizing vertebral members, and more particularly, to intervertebral implants, devices and methods of use in replacing an intervertebral disc, a vertebral member, or a combination of both to distract and/or stabilize the spine.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

As is known, various conditions and ailments may lead to damage of the spine, intervertebral discs and/or the vertebral members. The damage may result from a variety of causes including, but not limited to, events such as trauma, a degenerative condition, a tumor, or infection. Damage to the intervertebral discs and vertebral members can lead to pain, neurological deficit, and/or loss of motion.

Various procedures include replacing a section of or the entire vertebral member, a section of or the entire intervertebral disc, or both. One or more replacement implants may be inserted to replace the damaged vertebral members and/or discs. The implants are configured to be inserted into the intervertebral space and contact against adjacent vertebral members. The implants are intended to reduce or eliminate the pain and neurological deficit, and increase the range of motion.

The curvature of the spine and general shapes of the vertebral members may make it difficult for the implants to adequately contact the adjacent vertebral members or to position the adjacent vertebral members in a desired orientation. There is thus a need for implants or devices configurable to match the spinal anatomy for secure contact and/or desired orientation when implanted into an intervertebral space.

### SUMMARY

The present application discloses implants or devices for insertion into an intervertebral space between first and second vertebral members, the implant comprising an implant body with at least one base section which includes positioning teeth, and an end cap adapted for selective positioning at a selected point on the base section. The end cap comprises an exterior contact surface that faces away from the implant body when the end cap is positioned on the base section, a seating surface adapted to contact the base section when the end cap is positioned on the implant body, an end cap angulation which can be an angular value in the range of between zero degrees to fifteen degrees)(0°-15°, and positioning passages adapted to receive positioning teeth, the positioning passages and positioning teeth complimentarily configured to facilitate positioning the end cap on the base section. The implant also includes a locking mechanism adapted to lockingly engage the end cap and base section. The implant imparts the end cap angulation to an adjacent vertebral body at the selected point when the implant is positioned in the intervertebral space. The end cap can be selectively positioned on the base section via rotational adjustment of the end cap about an implant longitudinal axis so that the end cap angulation coincides to the selected point. The locking mechanism can comprise a base section locking surface disposed on the base section and an end cap locking surface disposed on the end cap wherein the base section locking surface and end cap locking surface have opposing and complimentary configurations that together provide a locking engagement when the end cap is positioned on the base section. The base section locking surface and end cap locking surface comprise tapered configurations.

There is also provided an implant for insertion into an intervertebral space between a first and second vertebral member comprising an implant body with at least one base section with positioning teeth and a male locking surface, and an end cap adapted for selective positioning at a selected point on the base section. The end cap comprises an exterior contact surface that faces away from the implant body when the end cap is positioned on the base section, a seating surface adapted to contact the base section when the end cap is positioned on the implant body, an end cap angulation having an angular value in the range of between zero degrees to fifteen degrees)(0°-15°, positioning passages extending between the exterior contact surface and the seating surface and adapted to receive positioning teeth, the positioning passages and positioning teeth complimentarily configured to facilitate positioning the end cap on the base section, and a female locking surface complimentarily configured to lockingly engage the end cap on the base section. The implant will impart the end cap angulation to an adjacent vertebral body at the selected point when the implant is positioned in the intervertebral space. The end cap is selectively positioned on the base section via rotational adjustment of the end cap about an implant longitudinal axis so that the end cap angulation coincides to the selected point. The male locking surface and female locking surface comprise a tapered configuration and have opposing and complimentary configurations that together provide a locking engagement when the end cap is positioned on the base section.

The various aspects of the various embodiments may be used alone or in any combination, as is desired.

### BRIEF DESCRIPTION OF THE DRAWINGS

FIG. 1 is a view of an implant positioned between vertebral members according to one embodiment;

FIG. 2 is a perspective view of an implant with end caps according to one embodiment;

FIG. 3 is an exploded perspective view of FIG. 2;

FIG. 4A is a section view along the line A-A of the implant base section and end cap of FIG. 2;

FIG. 4 is a top view of FIG. 2;

FIG. 5 is a partial side view of an implant with end caps according to another embodiment; and

FIG. 6 is an isometric view of an end cap according to another embodiment.

### DETAILED DESCRIPTION

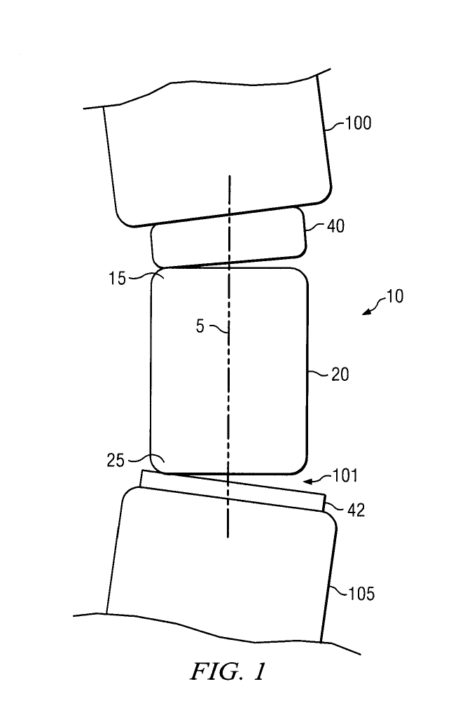

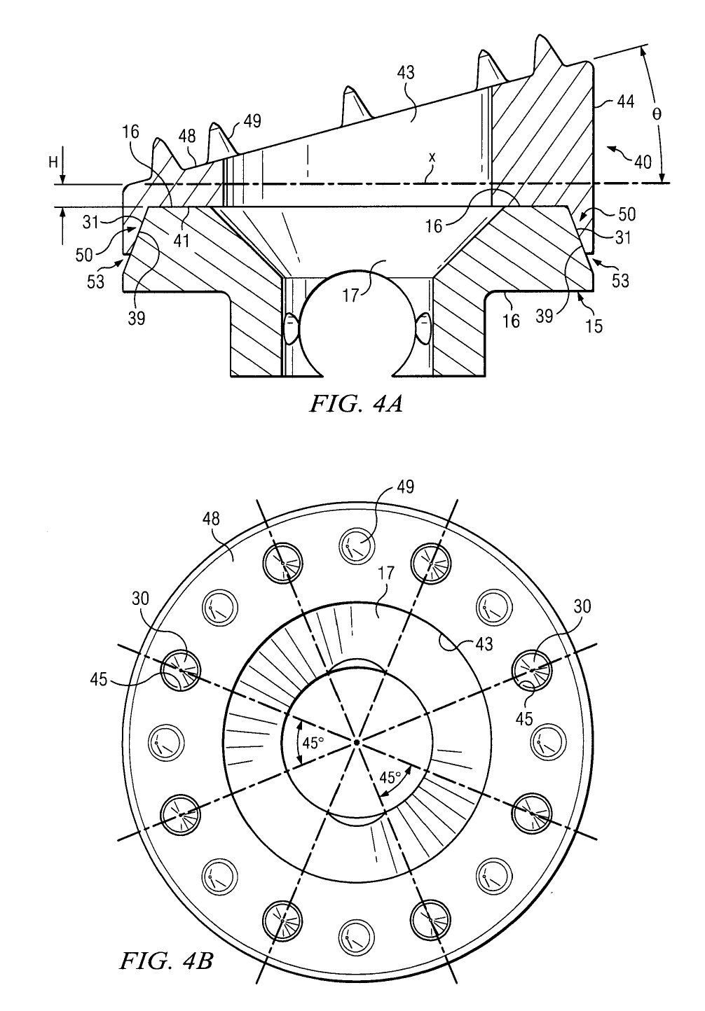

The present application is directed to intervertebral implants for spacing apart vertebral members 100 and 105. FIG. 1 shows an implant 10 positioned within an intervertebral space 101 formed between vertebral members 100 and 105. The implant 10 includes an implant body 20 and one or more end caps 40 and 42 attached to the implant body 20 at a first or second implant base section 15 and 25. The one or more end caps 40 and 42 will connect to the implant body 20 to impart a desired angulation θ (shown in FIG. 4A) to impart an angular orientation and/or position to an adjacent vertebral member 100 or 105. A connection or locking mechanism 50, shown in one embodiment in FIG. 4A as a taper locking mechanism, may connect an end cap 40 and 42 to the base section 15 and 25 to improve the contact and stability of the intervertebral implant 10 with the vertebral members 100 and 105 and drive angular orientation for correction or improved alignment of the spine.

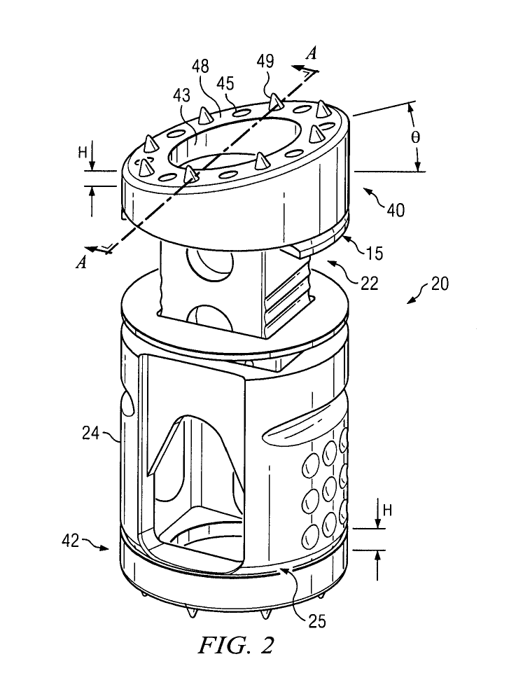

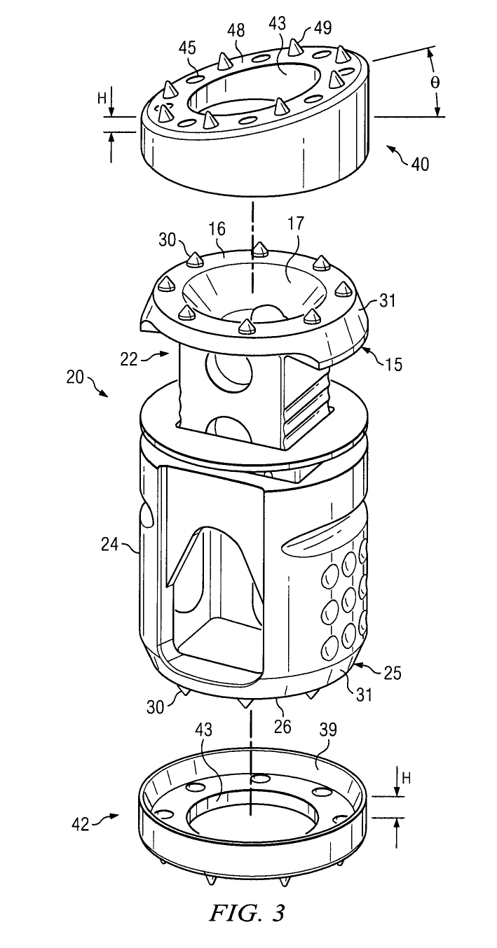

As shown in FIG. 1, the implant 10 may include first and second end caps 40 and 42 positioned at opposite ends of the implant body 20 at first and second base sections 15 and 25. The first and second end caps 40 or 42 may have the same or different configuration, height H, and/or the same or different end cap angulation θ. For example, as shown in FIGS. 2, 3 & 4A, one end cap 40 has an angulation θ of 15° degrees and a second end cap 42 has an angulation θ of zero degrees and an end cap height H. Those of skill in the art will recognize that one or two end caps 40 or 42 may be used during a medical procedure with the implant 10 to impart desired or needed height H and angulation θ of adjacent vertebral members 100 or 105 and to correct, improve and stabilize the affected spinal anatomy.

FIGS. 2 and 3 show an assembled and exploded perspective view of an implant 10 with upper and lower end caps 40 and 42 according to one embodiment. In the aspect shown in FIGS. 2 & 3, the implant body 20 is constructed of two relatively movable sections 22 and 24 to permit adjustment of the axial height of the implant 10. The implant body 20 includes an inner implant body 22 adapted to axially travel inside an outer implant body 24 to thereby enable selected or controlled collapse and expansion of the implant 10. The outer implant body 24 includes a hollow interior and the inner implant body 22 includes a first base section 15. The inner implant body 22 is sized to fit within and axially travel along the hollow interior of the outer implant body 24 to adjust the height of the implant body 20 along the longitudinal axis 5. Both the inner and outer implant bodies 22 and 24 may be hollow and include one or more apertures to receive bone growth material. Also, one or more apertures may extend through the body 20 walls to the hollow interior. However, the implant body 20 may also be constructed from a single section with a fixed height measured between the first and second base sections 15 and 25.

The implant body 20 is configured to receive the end cap 40 or 42. The inner implant body 22 includes a first base section 15 with positioning teeth or spikes 30 and a male locking surface 31, shown in FIGS. 3 & 4A. The positioning teeth or spikes 30 extend away from an exterior surface 16 of the first base section 15 in a substantially vertical or axial direction. The male locking surface 31 is preferably an inclined surface extending around the exterior periphery of the first base section 15. The outer implant body 24 includes a second base section 25 with similar positioning teeth or spikes 30 which extend away from an exterior surface 26 of the second base section 25 in a substantially vertical or axial direction, and a male locking surface 31 which is an inclined surface extending around the exterior periphery of the second base section 25. The implant base section 15 and 25 also includes a base aperture 17 to receive or permit delivery of bone growth material into the implant 10 which will permit fusion once the implant is in place between the vertebral members 100 and 105. The base aperture 17 is preferably adjacent and aligned with a corresponding end cap central aperture 43. Those of skill in the art will recognize that the base aperture 17 and end cap central apertures 43 may also be non-aligned if desired or needed by a surgeon, medical procedure or clinical application.

The implant base section 15 and 25 preferably comprises two or more positioning teeth or spikes 30 to accept and provide for stable placement and positioning of the end cap 40 or 42 onto the implant base section 15 and 25, via positioning with corresponding positioning apertures or passages 45 in the end cap 40 and 42. In one aspect shown in FIGS. 2-4B, the implant base section 15 and 25 will preferably have eight positioning teeth 30 to accept the placement of the end cap 40 or 42 onto the implant base section 15 and 25. The positioning teeth 30 are located around the periphery of the exterior surface 16 and 26 of the first and second base section 15 and 25. The positioning teeth 30 are preferably located evenly or equidistantly around the periphery of the exterior surface 16 and 26, as best shown in FIG. 4B. The positioning teeth 30 may also be located at non-equidistant locations around the exterior surface 16 and 26 as may be selected or required by a surgical procedure or clinical application, or surgeon need or selection. As will be discussed below, the number of positioning teeth 30 can be equal to or less than the number of end cap positioning apertures 45 to permit placement of the end cap 40 or 42 onto the implant base section 15 and 25 via positioning between the positioning teeth 30 and corresponding positioning apertures or passages 45, as shown in FIGS. 2 & 4B.

The positioning teeth 30, in addition to providing a mechanism to accept placement of the end cap 40 or 42 on the implant base section 15 and 25, also provide a securing and stabilizing function so that the end cap 40 or 42 can more securely rest on the implant base section 15 and 25. The greater the number of positioning teeth 30, the better the end cap 40 or 42 will rest and be positionable on the implant base section 15 and 24. In one aspect, the positioning teeth 30 may have a cone or conical configuration, shown in FIG. 3 to more easily permit the implant base section 15 and 25 to accept the positioning apertures 45 when the end cap 40 or 42 is placed on the implant base section 15 and 25. Those of skill in the art will recognize that other shapes or configurations may be used, depending on surgeon need, or procedure or clinical need, for the positioning teeth 30 which also permit the implant base section 15 and 25 to accept and align with the positioning apertures 45. The positioning teeth 30 may have different heights which permit the base teeth to extend through the exterior contact surface 48 or only extend partially into the positioning apertures 45 in the end cap 40 or 42 as might be needed or required by a surgeon or clinical procedure

The male locking surface 31 is preferably an inclined surface that extends around the periphery of the first base section 15 or 25 and enables or permits placement and locking of the end cap 40 or 42 onto the implant base section 15 or 25 via engagement of a corresponding complimentary female locking surface 39 on the end cap 40 or 42, shown in FIGS. 3 & 4A and discussed below. The complimentary male and female locking surfaces 31 and 39 engage to form a locking mechanism 50 when the end cap 40 or 42 is placed on the implant base section 15 or 25. In a preferred embodiment, the complimentary male and female locking surfaces 31 and 39 engage to form a tapered lock locking mechanism 50. The positioning teeth or spikes 30 permit stable and positional placement of the end cap 40 or 42 onto the implant base section 15 and 25, via alignment and positioning with corresponding positioning apertures or passages 45 on the end cap 40 and 42.

The complimentary male and female locking surfaces 31 and 39 will have complimentary configurations so that they may appropriately result in a locking engagement when the end cap 40 is positioned on the implant base section 15 and 25. As shown in FIGS. 3 and 4A, the complimentary male and female locking surfaces 31 and 39 have complimentary incline surfaces along the periphery of the end cap and implant base section 15 and 25 resulting in a taper lock upon placement of an end cap 40 onto the implant base section 15 or 25. Those of skill in the art will recognize that other sizes, shapes and configuration may be used for the male and female locking surfaces 31 and 39 depending on medical procedure or clinical need, or surgeon need or selection, as long as they have a complimentary configuration which permits insertion of and locking engagement of a male locking surface 31 with a female locking surface 39 when the end cap 40 is positioned on the implant base section 15 and 25.

In a preferred embodiment, the positioning teeth 30 have a height such that they extend partially into the end cap 40 or 42 body but do not extend past the exterior contact surface 48. The implant base section 15 or 25 may have two or more extending positioning teeth 30 to accept and provide for stable placement of the end cap 40 or 42 onto the implant base section 15 and 25, via alignment and positioning with corresponding end cap positioning apertures or passages 45. In such an embodiment, the corresponding end cap 40 will have positioning apertures 45 which partially extend into the end cap 40 and 42 and are configured to accept the positioning teeth 30. The implant base section 15 and 25 will have two or more positioning teeth 30 adapted to fit into corresponding end cap positioning apertures 45. The greater the number of positioning teeth 30, the more securely the end cap 40 or 42 will sit on the implant base section 15 and 25.

The end cap 40 or 42 may have one or more end cap teeth 49 which assist in providing a securing and stabilizing function of the combined end cap 40 or 42 and implant body 20. The end cap teeth 49 may be a series of equidistantly spaced end cap teeth 49 extending from the end cap exterior surface 48, for example as shown in FIGS. 2-4B. Those of skill in the art will recognize that the end cap teeth 49 may have an arrangement and number of end cap teeth 49 that may depend on the medical procedure, on clinical need, or surgeon need or selection. For example, the end cap teeth 49 may be a number of large end cap teeth, or a combination of large and small end cap teeth that extend from the end cap exterior surface 48 so long as they assist in providing a securing and stabilizing function of the combined end cap 40 or 42 and implant body 20. The end cap teeth 49 could also be a series or pattern of uniform teeth that cover the entire end cap exterior surface 48 and assist in providing a securing and stabilizing function of the combined end cap 40 or 42 and implant body 20.

The end cap 40 or 42 includes end cap teeth 49 which will come in contact with and engage the end plate of an adjacent vertebral body once the combined implant body 10 and end cap 40 or 42 is positioned in an intervertebral space 101 between the vertebral members 100 and 105. The end cap teeth 49 will provide a securing and stabilizing function of the combined end cap 40 or 42 and implant body 10. The end cap teeth 49 will extend from the end cap exterior surface 48 sufficiently to penetrate, insert and embed into the adjacent vertebral member 100 and 105 end plate. This end cap teeth aspect provides stability for the expanded implant 10 and prevents the inserted implant 10, which includes the implant body 10 and end cap combination with gripping end cap teeth 49, from being ejected out of the intervertebral space 101 after implant 10 insertion. The actual end cap teeth 49 height can vary to accommodate the selection or need of a surgeon, medical procedure or clinical need. When an implant 10 with positioning base teeth and one or two end caps 40 or 42 is inserted into an intervertebral space 101 and set to a desired implant height in the intervertebral space 101, via appropriate instruments (not shown), the protruding end cap teeth 49 will penetrate and insert into the adjacent vertebral member 100 and 105 end plate to maintain a stable implant 10 position between the adjacent vertebral members 100 and 105.

FIG. 2 is a perspective view of the implant with end caps according to one embodiment. FIG. 4A is a section view along the line A-A of the implant base section 15 and end cap 40 of FIG. 2. As discussed previously, the implant base section 15 comprises positioning teeth or spikes 30 and a male locking surface 31. The positioning teeth or spikes 30 provide for stable placement and positioning of the end cap 40 or 42 onto the implant base section 15 via alignment and positioning with corresponding end cap positioning apertures or passages 45. In one aspect shown in FIGS. 2, 3 & 4B, the implant base section 15 preferably has eight positioning teeth 30 to accept placement of the end cap 40 onto the implant base section 15. The male locking surface 31 is preferably an inclined surface that extends around the periphery of the first base section 15 or 25 and enables or permits placement and locking of the end cap 40 or 42 onto the implant base section 15 or 25 via engagement of a corresponding complimentary female locking surface 39 on the end cap 40 or 42, shown in FIGS. 3 & 4A. The complimentary male and female locking surfaces 31 and 39 engage to form a taper locking mechanism 50 when the end cap 40 or 42 is placed on the implant base section 15 or 25.

The end cap 40 comprises an annular-like shape with an exterior contact surface 48, positioning apertures or passages 45, a seating surface 41, a female locking surface 39, a substantially vertical exterior cap wall 44 and a central aperture 43. The exterior contact surface 48 and a seating surface 41 are bounded by the vertical exterior cap wall 44 and the central aperture 43. The central aperture 43 is preferably aligned with and the same size as the corresponding base aperture 17. Those of skill in the art will recognize that the central aperture 43 and base aperture 17 may also be of different sizes and non-aligned if desired or needed by a surgeon, medical procedure or clinical application.

The contact surface 48 extends around the central opening 43. The contact surface 48 includes end cap teeth 49 which will engage the end plates of an adjacent vertebral member 100 or 105 to assist the implant 10 grip the vertebral member end plate, provide implant 10 stability in the disc space 101, and prevent implant 10 ejection from the intervertebral space 101. Those of skill in the art will recognize that the number, size, shape, orientation and spacing of the end cap teeth 49 may vary according to the needs of a medical procedure or clinical application.

The seating surface 41 has a configuration that complements the exterior surface 16 of the implant base section 15 such that the end cap 40 can seat on the implant base section 15 when the end cap 40 is positioned and locked onto the base section. In a preferred aspect, the seating surface 41 and the exterior surface 16 have complimentary and substantially flat surfaces such that the end cap 40 can seat flush on the implant base section 15. When the end cap 40 is positioned on the implant base section 15, the positioning teeth 30, which extend from the exterior surface 16, provide for a stable placement and positioning of the end cap 40 onto the implant base section 15 via alignment and positioning with corresponding positioning apertures or passages 45 in the end cap 40. In the aspect shown in FIGS. 2-4B, the implant base section 15 has eight positioning teeth 30 that extend from the exterior surface 16 to accept placement of the end cap 40. The positioning teeth 30, in addition to providing a mechanism to accept placement of the end cap 40 on the implant base section 15, also provide a securing and stabilizing function so that the end cap 40 or 42 can more securely rest on the implant base section 15 and 25. In a preferred aspect, the positioning teeth 30 extend partially into the end cap 40 body but do not extend past the exterior contact surface 48. In such an embodiment, the corresponding end cap 40 will have complimentary positioning apertures 45 which extend into the end cap body and are configured to accept the positioning teeth 30.

Additionally, when the end cap 40 is positioned on the implant base section 15, the complimentary male and female locking surfaces 31 and 39, which have complimentary configurations, lockingly engage. As shown in FIGS. 3 and 4A, the complimentary male and female locking surfaces 31 and 39 have complimentary inclined surfaces along the periphery of the end cap and implant base section 15 which lockingly engage and resulting in a taper lock upon placement of an end cap 40 onto the implant base section 15. Those of skill in the art will recognize that other sizes, shapes and configuration may be used for the male and female locking surfaces 31 and 39 depending on medical procedure or clinical need, or surgeon need or selection, as long as they have a complimentary configuration which permits insertion of and locking engagement of a male locking surface 31 with a female locking surface 39 when the end cap 40 is positioned on the implant base section 15.

The end cap 40 comprises one or more end cap teeth 49 which assist in providing a securing and stabilizing function of a combined end cap 40 and implant body 20. The end cap teeth 49 may be a series of equidistantly spaced end cap teeth 49 extending from the end cap exterior surface 48, for example as shown in FIGS. 2-4B. Those of skill in the art will recognize that the end cap teeth 49 may have an arrangement and number of end cap teeth 49 that may depend on the medical procedure, on clinical need, or surgeon need or selection. For example, the end cap teeth 40 may be a number of large end cap teeth, or a combination of large and small end cap teeth that extend from the end cap exterior surface 48 so long as they assist in providing a securing and stabilizing function of the combined end cap 40 or 42 and implant body 20. The end cap teeth 49 could also be a series or pattern of uniform teeth that cover the entire end cap exterior surface 48 and assist in providing a securing and stabilizing function of the combined end cap 40 or 42 and implant body 20. The end cap teeth 49 may also comprise a combination of large end cap teeth and a series of smaller end cap teeth 49 extending from the end cap exterior surface 48. The actual end cap teeth height may vary to accommodate the selection or need of a surgeon, medical procedure or clinical need.

The end cap teeth 49 will engage the end plate of an adjacent vertebral body once the combined implant body 10 and end cap 40 and/or 42 are positioned in an intervertebral space 101. The end cap teeth 49 will come in contact with and engage the end plate of an adjacent vertebral body and provide a securing and stabilizing function of the combined end cap 40 and implant body 10. The end cap teeth 49 will extend from the end cap exterior surface 48 sufficiently to grip, penetrate, insert and embed into the adjacent vertebral member 100 and 105 end plate to thereby provide implant stability in the intervertebral disc space 101 and prevent the inserted implant 10 from being ejected out of the intervertebral space 101 after implant 10 insertion. The actual end cap teeth 49 height can vary to accommodate the selection or need of a surgeon, medical procedure or clinical need. When an implant 10, with positioning base teeth 30 and one or two end caps 40 or 42, is inserted into an intervertebral space 101 and set to a desired implant height, via appropriate instruments (not shown), the protruding end cap teeth 49 will penetrate and insert into the adjacent vertebral member end plate to maintain a stable implant 10 position between the adjacent vertebral members 100 and 105.

The end cap 40 further comprises an angulation θ aspect and an end cap vertex height H. The end cap angulation θ and cap height H may have a range of values as may be selected or needed by a surgeon, medical procedure or clinical application. In one aspect, preferred discrete values for end cap angulation are 0°, 4°, 8° and 15° degrees measured from an angulation reference line X, shown in FIG. 4A. In other embodiments, the preferred angulation θ values may be in the range of zero and thirty degrees)(0°-30°, with a preferred range of between zero and fifteen degrees)(0°-15°. In one aspect, the cap height H may have preferred values in 1.0 mm or 0.5 mm increments measured from the end cap seating surface 41. The angulation reference line X is preferably at the cap height H value as shown in FIG. 4A. The end cap's angulation θ is a measure of the exterior contact surface's 48 inclination relative to the angulation reference line X. Insertion of an implant 10 with an end cap 40 having an angulation θ aspect enables the end cap 40 to impart a desired or selected angulation θ to an adjacent vertebral member 100 or 105. In this manner, selective angulation θ can be imparted to the adjacent vertebral body 100 or 105 and thereby assist in the correction and/or improved orientation, stabilization and alignment of the spine. In the event where additional implant height H is desired or required without any angulation, an end cap 40 having angulation θ of 0° degrees may be used to impart the additional height to the implant 10 in the amount of an end cap height H. Such a case is illustrated in FIGS. 2 and 3 for a lower end cap 42 attached to the outer implant body 24 which show an isometric view of an implant base section 25 with an end cap 42 having angulation θ of 0° degrees and a certain cap height H.

The end cap 40 or 42 preferably comprises two or more positioning apertures or passages to permit positioning and insertion of positioning teeth 30 into the positioning apertures 40 or 42 when the end cap 40 or 42 is positioned on the implant base section 15 and 25. The end cap 40 positioning apertures or passages 45 extend in a substantially vertical orientation between and through the exterior contact surface 48 and seating surface 41. In another embodiment, the end cap 40 positioning apertures or passages 45 may extend away from the seating surface 41 in a substantially vertical orientation into the end cap body, and toward the exterior contact surface 48 but do not extend to the exterior contact surface 48 of the end cap. The positioning apertures or passages 45 will have a size and configuration which permits positioning and insertion of positioning teeth 30 into the positioning apertures 45 when the end cap 40 is positioned on the implant base section 15 and 25. Those of skill in the art will recognize that different sizes, shapes and configuration may be used for the positioning apertures or passages 45 depending on medical procedure or clinical need, or surgeon need or selection, as long as they permit positioning and insertion of positioning teeth 30 into the positioning apertures 40 or 42 when the end cap 40 or 42 is positioned on the implant base section 15 and 25.

The positioning apertures or passages 45 are located in an end cap area between the vertical exterior cap wall 44 and the central aperture 43, and between the vertical exterior cap wall 44 and the central aperture 43. The end cap positioning aperture or passage 45 may extend between and through the contact surface 48 and seating surface 41, or may extend to a point between the contact surface 48 and seating surface 41, depending on the height of the positioning teeth used. The end cap 40 preferably has at least the same number of positioning apertures 45 as the number of extending positioning teeth 30 since the positioning apertures or passages 45 accept entry of extending positioning teeth 30 when the end cap 40 or 42 is placed on the implant base section 15 and 25. The positioning apertures or passages 45 are preferably spaced and located evenly or equidistantly around the end cap area between the vertical exterior cap wall 44 and the central aperture 43. The positioning teeth 30 are preferably complimentarily located evenly or equidistantly around the periphery of the exterior surface 16 and 26, as best shown in FIG. 4B. The spacing and location of the positioning apertures 45 is selected to complimentarily align with the spacing and location of the positioning teeth 30 to permit end cap 40 insertion and positioning with the positioning teeth 30 of the implant base section 15 or 25. Those of skill in the art will recognize that the positioning aperture or passages 45 may also be spaced or located at non-equidistant points around the end cap area between the vertical exterior cap wall 44 and the central aperture 43 as may be desired or required by a surgical procedure or clinical application, or surgeon need or selection. However, in the non-equidistant spacing or locating case, the positioning apertures 45 must still be spaced and located to complimentarily align with the spacing and location of the positioning teeth 30 to permit end cap 40 positioning and insertion with the positioning teeth 30 of the implant base section 15 or 25.

As the positioning apertures or passages 45 accept entry of the positioning teeth 30, the end cap 40 must have at least the same number of positioning apertures 45 as the number of positioning base teeth 30. For example, in the case of FIG. 4B, the end cap 40 has eight positioning apertures or passages 45 and the base section 15 has eight positioning base teeth 30. If there are two positioning teeth, then the there must be at least two positioning apertures 45. If there are four positioning teeth 30 then there must be at least four positioning apertures or passages 45 in order that the end cap 40 can be inserted and seated onto the implant base section 15. The end cap 40 may also have more positioning apertures 45 than implant base section positioning teeth 30 which will provide a greater degree of rotation and positional control of placement of the end cap 40 on the implant base section, as discussed below.

The end cap 40 or 42 preferably comprises a female locking surface 39 which is preferably an inclined surface that extends away from the end cap seating surface 41 and around the periphery of the seating surface 41. The female locking surface 39 enables or permits placement and locking of the end cap 40 or 42 onto the implant base section 15 or 25 via engagement of a corresponding complimentary male locking surface 31 on the implant base section 15 or 25, shown in FIGS. 3 & 4A. The complimentary male and female locking surfaces 31 and 39 engage to form a locking mechanism 50 when the end cap 40 or 42 is placed on the implant base section 15 or 25. In a preferred embodiment, the complimentary male and female locking surfaces 31 and 39 engage to form a tapered lock locking mechanism 50. The positioning apertures or passages 45 on the end cap 40 and 42 simultaneously permit stable and positional placement of the end cap 40 or 42 onto the implant base section 15 and 25 via positioning with corresponding positioning teeth or spikes 30.

The complimentary male and female locking surfaces 31 and 39 will have a complimentary configuration so that they may appropriately result in a locking engagement when the end cap 40 is positioned on the implant base section 15 and 25. As shown in FIGS. 3 and 4A, the complimentary male and female locking surfaces 31 and 39 preferably have complimentary incline surfaces along the periphery of the end cap and implant base section 15 and 25 resulting in a taper lock upon placement of an end cap 40 onto the implant base section 15 or 25. The complimentary male and female locking surfaces 31 and 39, when lockingly engaged, result in a locking engagement sufficient to hold the end cap 40 or 42 on the implant base plate 15 or 25 to permit a surgeon to pre-assemble the end cap 40 or 42 to an implant 10 for insertion into an intervertrebal disc space 101. As long as there is a complimentary angled or tapered relationship 53 between the complimentary male and female locking surfaces 31 and 39, the end cap 40 and 42 and the implant base plate 15 or 25 will be securely locked together in place when the end cap 40 or 42 is positioned on the implant base section 15 and 25 via a self-locking press fit arrangement, interference fit, flush friction fit arrangement, friction fit or compression fit. Those of skill in the art will recognize that other sizes, shapes and configuration may be used for the male and female locking surfaces 31 and 39 depending on medical procedure or clinical need, or surgeon need or selection, as long as they have a complimentary configurations which permits insertion of and locking engagement of a female locking surface 39 with a male locking surface 31 when the end cap 40 is positioned on the implant base section 15 and 25.

The positioning apertures or passages 45 enable the end cap 40 to be selectively and rotationally positioned or adjusted on the implant base section 15 or 25. The end cap 40 or 42 is rotationally adjustable relative to the implant body 20 and implant base section 15 or 25 about the longitudinal axis 5 of the implant body 20. The number of positioning apertures 45 determine the number of positions or rotational positions at which the end cap 40 can be placed in or located on the implant base section 15 and 25. The greater the number of positioning apertures 45 the larger the number of positions or rotational positions the end cap 40 can be adjusted and placed in or located on the implant base section 15 and 25. The more positioning apertures or passages 45 there are, the greater degree of choice and control a surgeon will have in selecting a rotational position for the end cap 40 to be placed in or located on the implant base section 15 and 25. This aspect advantageously provides a surgeon selective control of where the end cap angulation θ and the end cap vertex height H will be rotational positioned on the implant base section 15 or 25. The ability to selectively position the end cap angulation θ permits a surgeon to determine where the end cap angulation θ and end cap height H will be applied or imparted to an the adjacent vertebral body 100 or 105. Prior to insertion of the implant 10 into the intervertebral disc space 101, the surgeon can decide where the end cap angulation θ and the end cap vertex height H are desired or needed for a particular medical procedure or clinical application.

As noted previously, a surgeon can selectively position the end cap 40 on the implant base plate 15 or 25 by rotating the end cap 40, either clockwise or counterclockwise, and then inserting the end cap positioning apertures 45 onto the positioning teeth 30 at the desired or needed rotational position on the implant base plate 15. This aspect enables selective positioning of the end cap angulation θ which in turn permits the surgeon to decide where the end cap angulation θ and end cap height H will be applied or imparted to an the adjacent vertebral body 100 or 105. The clockwise or counterclockwise rotation of the end cap 40 moves or adjusts the end cap's 40 angulation θ and the end cap vertex height H relative to the implant base section 15 so as to position the end cap angulation θ and vertex height H at a desired or required point on the implant base section 15 or 25. For example at anterior, antereolateral, posterior or lateral points about the vertebral member 100 or 105, or vertebral disk space 101. This is in turn will position the end cap angulation θ and vertex height H at a desired or required point relative to the adjacent intervetebral member 100 or 105 once the implant 10 is inserted and positioned within the intervertebral space 101. The end cap 40 will then be able to impart desired or required angulation θ, orientation and vertex height H on the adjacent vertebral body at selected or required points on the adjacent vertebral body 100 or 105 to correct or improve the angulation, orientation, alignment and stabilization of the spine or spinal anatomy.

As noted above, the end cap 40 may be rotated so as to contact and impart angulation θ at different location points about the periphery of the adjacent vertebral body 100 or 105. The number of positioning aperture or passages 45 impact the incremental degree of control, through clockwise or counterclockwise end cap 40 rotation, that a surgeon will have in selecting the end cap angulation θ position between the implant 10 and the adjacent vertebral body 100 or 105. In the embodiment shown in FIG. 4B, the end cap 40 has eight positioning apertures 45 which are evenly or equidistantly space in the area between the contact surface 48 and seating surface 41. The equidistant spacing results in the positioning apertures 45 being located and spaced apart from each other at about forty-five degrees (45°) around the end cap 40 as shown in FIGS. 2-4B. In this embodiment then, the end cap 40 can be rotationally advanced, clockwise or counterclockwise, in single or multiple increments of forty-five degrees (45°) in order to rotationally position or reposition the end cap angulation θ position between the implant 10 and the adjacent vertebral body 100 or 105.

A greater degree of control in rotationally and incrementally advancing, clockwise or counterclockwise, the end cap 40 about the implant base section 15 may be obtained by increasing the number of positioning apertures or passages 45. For example, if the end cap 40 were to have sixteen (16) positioning apertures or passages 45 evenly or equidistantly spaced in the area between the contact surface 48 and seating surface 41. Then, equidistant and substantially circular spacing would result in the positioning apertures or passages 45 being located and spaced apart from each other at twenty-two and a half degrees (22.5°) around the substantially circular area between the contact surface 48 and seating surface 41 of the end cap 40. In this case, the end cap 40 can be rotationally advanced, clockwise or counterclockwise, in single or multiple increments of twenty-two and a half degrees (22.5°) in order to position or reposition the end cap angulation θ position between the implant 10 and the adjacent vertebral body 100 or 105. The larger number of positioning apertures or passages 45 provides a surgeon the ability to rotationally position or reposition the end cap 40 in smaller discrete increments. This greater degree of control provides the surgeon with more precise control on where the end cap angulation θ will be positioned between the implant 10 and the adjacent vertebral body 100 or 105. In this manner, the selected angulation θ and end cap vertex height H can be imparted to an adjacent vertebral member 100 or 105 to thereby impart or drive angular orientation and height adjustment of the adjacent vertebral member 100 or 105 for correction or improved alignment, angulation, orientation, and stabilization of the spine or spinal anatomy.

Once the appropriate or desired rotational position of the end cap 40 or 42 is obtained, the surgeon inserts or places the end cap 40 or 42 so that the end cap 40 engages and locks onto the implant base plate 15 or 25, as shown in FIGS. 2 & 4A. The locking of the end cap 40 onto the implant base plate 15 or 25 is accomplished through a connection or locking mechanism 50 where the complimentary male and female locking surfaces 31 and 39 will complimentary interact and lockingly engagement when the end cap 40 is positioned on the implant base section 15 and 25. The positioning apertures or passages 45 permit alignment, positioning and insertion of corresponding positioning teeth 30 on the implant base plate 15 or 25. As shown in FIGS. 2 and 4A, the complimentary male and female locking surfaces 31 and 39, when engaged, result in a locking engagement sufficient to hold and securely lock the end cap 40 or 42 on the implant base plate 15 or 25. In this embodiment, when the complimentary female locking surface 39 is positioned and forced onto the complimentary male locking surfaces 31, the resulting force experienced between the pressed adjacent tapered surfaces provides secure locking between the end cap 40 and the implant base plate 15 or 25 via a taper locking mechanism 50.

The locking mechanism 50 comprises male and female locking surfaces 31 and 39 having complimentary configurations that when engaged result in a locking engagement when the end cap 40 is positioned on the implant base section 15 and 25. In this manner, the locking mechanism 50 can sufficiently hold the end cap 40 or 42 on the implant base plate 15 or 25 to permit a surgeon to pre-assemble the end cap 40 or 42 to an implant 10 prior to insertion into an intervertrebal disc space 101. As long as there is a complimentary angled or tapered relationship 53 between the complimentary male and female locking surfaces 31 and 39, the end cap 40 and 42 and the implant base plate 15 or 25 will be securely locked together in place when the end cap 40 or 42 comes down on the implant base section 15 and 25 via a self-locking press fit arrangement, interference fit, flush friction fit arrangement, friction fit or compression fit. Those of skill in the art will recognize that other sizes, shapes and configurations may be used for the positioning apertures and passages 45 and positioning teeth 30, and male and female locking surfaces 31 and 39 to form the locking mechanism 50 depending on medical procedure or clinical need, or surgeon need or selection, so long as they have complimentary arrangement and configurations which permit locking engagement between the male and female locking surfaces 31 and 39 when the end cap 40 is positioned on the implant base section 15 and 25 and positioning and insertion of the positioning teeth 30 into the positioning locking apertures 45.

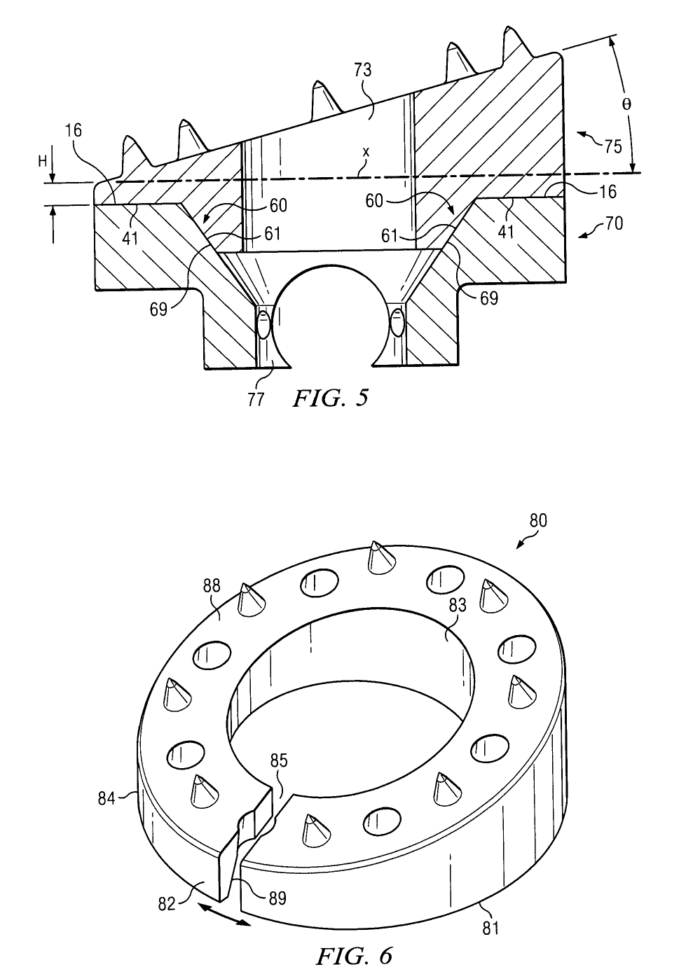

FIG. 5 is a partial side section view of a modified implant base section 70 and an end cap 75 according to another embodiment. The modified base section 70 and end cap 75 are similar in structure, form and function to the implant 10 and end caps 40 and 42 already discussed with respect to FIGS. 1-4B, with the exception that the modified locking mechanism 60 is now located or positioned at or near the central apertures 77 and 73 of the modified implant base section 70 and end caps 75, respectively. In this embodiment, the male locking surface 69 extends from the end cap 75 about the central aperture 73, while the female locking surface 61 is located on the inside surface of implant's base section central aperture 77.

The implant base section 70 is configured to receive the end cap 75. The implant base section 70 includes a base aperture 77 which will preferably align with a corresponding end cap central aperture 73. The implant base section 70 includes positioning teeth or spikes (not shown) and a female locking surface 61. As with the prior discussed embodiments, the positioning teeth will permit stable and positional placement of the end cap 75 onto the implant base section 70 via alignment and positioning with corresponding positioning apertures or passages (not shown) on the end cap 75. The positioning teeth or spikes though not shown can be substantially similar in form and function to the positioning teeth and spikes 30 previously described and discussed with respect to FIGS. 1-4B. The female locking surface 61 is preferably an inclined surface which extends away from the exterior surface 16 and toward the base aperture 77 of the implant base section 70, and which also extends around the interior periphery of implant base section's central aperture 77.

The end cap 75 comprises an annular-like shape with an exterior contact surface, positioning apertures or passages (not shown), a seating surface 41, a male locking surface 69, a substantially vertical exterior cap wall and a central aperture 73. The central aperture 73 is preferably aligned with the corresponding implant base aperture 77. The seating surface 41 has a configuration that complements the exterior surface 16 of the implant base section 70 such that the end cap 75 can seat on the implant base section 70 when the end cap 75 is positioned and locked onto the base section. The seating surface 41 and the exterior surface 16 have complimentary and substantially flat surfaces such that the end cap 75 can seat flush on the implant base section 70. When the end cap 75 is positioned on the implant base section 70, the positioning teeth (not shown), will provide for stable placement and positioning of the end cap 75 onto the implant base section 70 via positioning with corresponding positioning apertures or passages (not shown) in the end cap 75.

The male locking surface 69 is preferably an inclined surface that extends away from the end cap seating surface 41 in an peripheral exterior surface around the end cap's central opening 73 which enables or permits placement and locking of the end cap 75 onto the implant base section 70 via engagement of the corresponding complimentary female locking surface 61 on the implant base section 70. The complimentary male and female locking surfaces 69 and 61 engage to form a locking mechanism 60 when the end cap 75 is placed on the implant base section 70. In a preferred embodiment, the complimentary male and female locking surfaces 69 and 61 engage to form a tapered lock locking mechanism 60 when the end cap 40 is positioned on the implant base section 15. Those of skill in the art will recognize that other sizes, shapes and configuration may be used for the male and female locking surfaces 69 and 61 depending on medical procedure or clinical need, or surgeon need or selection, as long as they have a complimentary configuration which permits insertion of and locking engagement of a male locking surface 69 with a female locking surface 61 when the end cap 40 is positioned on the implant base section 15. Additionally, the positioning teeth (not shown) will permit stable and positional placement of the end cap 75 onto the implant base section 70 via alignment and positioning with corresponding positioning apertures or passages (not shown) on the end cap 75.

As with the prior embodiments discussed with respect to FIGS. 2-4B, the end cap 75 comprises an angulation θ aspect and an end cap vertex height H. A surgeon can selectively position the end cap 75 on the implant base plate 70 by rotating the end cap 75, either clockwise or counterclockwise, and then inserting the end cap positioning apertures onto the positioning teeth at the desired or needed rotational position on the implant base plate 70. This aspect enables selective positioning of the end cap 75 angulation θ which permits the surgeon to decide where the end cap angulation θ and end cap height H will be applied or imparted to an the adjacent vertebral body 100 or 105. The clockwise or counterclockwise rotation of the end cap 40 moves or adjusts the end cap's angulation θ and the end cap vertex height H relative to the implant base section 70 so as to position the end cap angulation θ and vertex height H at a desired or required point on the implant base section 70. The end cap 75 can impart end cap angulation θ, orientation and vertex height H on the adjacent vertebral body at selected or required points on the adjacent vertebral body 100 or 105 to correct or improve the angulation, orientation, alignment and stabilization of the spine or spinal anatomy.

The locking mechanism 60 comprises male and female locking surfaces 69 and 61 having complimentary configurations that result in a locking engagement when the end cap 75 is positioned on the implant base section 70. The locking mechanism 60 will be able to sufficiently hold the end cap 75 on the implant base plate 70 to permit a surgeon to pre-assemble the end cap 75 to an implant prior to insertion into an intervertrebal disc space 101. As long as there is a complimentary angled or tapered relationship between the complimentary male and female locking surfaces 69 and 61, the end cap 75 and the implant base plate 70 will be securely locked together in place when the end cap 75 comes down onto the implant base section 70 via a self-locking press fit arrangement, interference fit, flush friction fit arrangement, friction fit or compression fit. Those of skill in the art will recognize that other sizes, shapes and configurations may be used for the positioning apertures and passages and positioning teeth, and male and female locking surfaces 69 and 61 to form the locking mechanism 60 depending on medical procedure or clinical need, or surgeon need or selection, so long as they have complimentary arrangement and configurations which permit positioning and insertion of the positioning teeth and positioning locking apertures, and locking engagement between the male and female locking surfaces 69 and 61 when the end cap 75 is positioned on the implant base section 70.

The complimentary male and female locking surfaces 69 and 61 will have complimentary configurations so that they may appropriately result in a locking engagement when the end cap 75 is positioned on the implant base section 70. As shown in FIG. 5, the complimentary male and female locking surfaces 69 and 61 have complimentary inclined surfaces located or positioned about or near the central apertures 77 and 73 of the modified implant base section 70 and end caps 75 resulting in a taper lock upon placement of an end cap 75 onto the implant base section 70. Those of skill in the art will recognize that other sizes, shapes and configuration may be used for the male and female locking surfaces 69 and 61 depending on medical procedure or clinical need, or surgeon need or selection, as long as they have a complimentary configuration which permits insertion of and locking engagement of a male locking surface 69 with a female locking surface 61 when the end cap 75 is positioned on the implant base section 70.

FIG. 6 is an isometric view of an end cap 80 according to another embodiment. The end cap 80 comprises a flexing slot 85 preferably located or positioned on the vertex side 82 of the end cap 80. The flexing slot 85 is a passage that is bounded by an exterior contact surface 88, a seating surface 41, a vertical exterior end cap wall 84 and a central aperture 83. The flexible slot 85 permits the endcap 80 to have a resilient or spring aspect which permits the end cap 80 to deflect and expand as may be necessary when the end cap 80 is positioned on an implant base, such as the implant bases discussed with respect to FIGS. 2-5. The flexible slot 85 with the resilient or spring aspect can be advantageous to accommodate variations and differences in dimensions and tolerances between complimentary angled or tapered locking surfaces, such as the female locking surface 89 and a corresponding male locking surface such as, in one case, the male locking surface 31 shown in FIGS. 3 & 4A. The flexible slot's 85 deflection and expansion properties permit the complimentary angled or tapered locking surfaces, between the end cap 80 and implant base, to retain the ability to properly and lockingly engage when the dimensions and tolerances between the locking mechanism's complimentary angled or tapered locking surfaces are not exact or have variance between them. The resilient or spring aspect of the slotted end cap 80 also advantageously imparts a friction holding force on the complimentary angled or tapered locking surfaces of the locking mechanism between the end cap 80 and implant base when the end cap 80 is positioned on a base section. The friction holding force results from the tendency of the flexible slot 85 to bias back to its undeflected, unexpanded or equilibrium position. Since the end cap 80 is biased to return to its undeflected equilibrium position, the female locking surface 89 would encounter a complimentary and opposing tapered male locking surface which would impart a friction holding force between the complimentary angled or tapered locking surfaces. The friction holding force aspect of the end cap 80 will thereby supplement and strengthen or fortify the locking engagement between the complimentary angled or tapered locking surfaces when the end cap 80 is positioned on a base section.

FIG. 6 depicts an endcap 80 which preferably comprises a female locking surface 89 which is preferably an inclined surface that extends away from the end cap seating surface 81 and around the periphery of the seating surface 41. In another aspect, the flexible slot 85 may also be used in an end cap with a male locking surface which extends from the end cap about the central aperture, similar to that shown in FIG. 5. Such an alternative slotted end cap would have aspects and properties such that it would have a resilient or spring aspect which permits the end cap to deflect as may be necessary when the end cap is positioned on an implant base. The flexible slot's deflection and expansion properties permit the complimentary angled or tapered locking surfaces, between the end cap and implant base, to retain the ability to properly and lockingly engage when the dimensions and tolerances between the locking mechanism's complimentary angled or tapered locking surfaces are not exact or have variance between them. This alternative slotted end cap would also advantageously impart a friction holding force on the complimentary angled or tapered locking surfaces of the locking mechanism due to the tendency of the flexible slot 85 to bias or return to its undeflected or equilibrium position. The friction holding force aspect imparted by the end cap flexible slot will thereby supplement and strengthen or fortify the locking engagement and taper lock between the complimentary angled or tapered locking surfaces when the end cap 80 is positioned on a base section.

The implants 10 and end caps 40, 42, 75 and 80 may be implanted within a living patient for the treatment of various spinal disorders. The implant 10 may also be implanted in a non-living situation, such as within a cadaver, model, and the like. The non-living situation may be for one or more of testing, training, and demonstration purposes.

The end caps disclosed in this disclosure are preferably comprised of biocompatible materials substrates which can be used in combination with implants or devices configured to be inserted into an intervertebral space and contact against adjacent vertebral members. The biocompatible material substrate may include, among others, polyetheretherketone (PEEK) polymer material, homopolymers, co-polymers and oligomers of polyhydroxy acids, polyesters, polyorthoesters, polyanhydrides, polydioxanone, polydioxanediones, polyesteramides, polyamino acids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof. The biocompatible material substrate may also be a metallic material and may include, among others, stainless steel, titanium, nitinol, platinum, tungsten, silver, palladium, cobalt chrome alloys, shape memory nitinol and mixtures thereof. The biocompatible material used can depend on the patient's need and physician requirements.

While embodiments of the invention have been illustrated and described in the present disclosure, the disclosure is to be considered as illustrative and not restrictive in character. The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An implant for insertion into an intervertebral space between a first and second vertebral member, the implant comprising:
an implant body with at least one base section, the base section including positioning teeth; and
an end cap adapted for selective positioning at a selected point on the base section, the end cap comprising
an exterior contact surface that faces away from the implant body when the end cap is positioned on the base section,
a seating surface adapted to contact the base section when the end cap is positioned on the implant body,
an end cap angulation, and
positioning passages adapted to receive positioning teeth, the positioning passages and positioning teeth complimentarily configured to facilitate positioning the end cap on the base section; and
a locking mechanism adapted to lockingly engage the end cap and base section;
wherein the implant imparts the end cap angulation to an adjacent vertebral body at the selected point when the implant is positioned in the intervertebral space.

2. The implant of claim 1, wherein the locking mechanism comprises
a base section locking surface disposed on the base section; and
an end cap locking surface disposed on the endcap;
wherein the base section locking surface and end cap locking surface have opposing and complimentary configurations that together provide a locking engagement when the end cap is positioned on the base section.

3. The implant of claim 2, wherein the base section locking surface and end cap locking surface comprise tapered configurations.

4. The implant of claim 3, wherein
the base section locking surface is a male locking surface, and
the end cap locking surface is a female locking surface.

5. The implant of claim 3, wherein
the base section locking surface is a female locking surface, and
the end cap locking surface is a male locking surface.

6. The implant of claim 1, wherein the end cap is selectively positioned on the base section via rotational adjustment of the end cap about an implant longitudinal axis so that the end cap angulation coincides to the selected point.

7. The implant of claim 1, wherein the end cap angulation comprises an angular value in the range of between zero degrees to fifteen degrees (0°-15°.

8. The implant of claim 1, wherein the end cap further comprises an end cap height measured relative to the seating surface which enables the implant to both impart end cap height and end cap angulation to the adjacent vertebral body at the selected point.

9. The implant of claim 1, wherein the positioning passages are spaced and located to enable complimentarily alignment with the positioning teeth to permit the end cap to be selectively positioned on the base section.

10. The implant of claim 2, wherein the end cap further comprises a flexible slot adapted to permit the endcap to deflect when the end cap is positioned on the base section and thereby impart a friction holding force on the complimentary base section locking surface and end cap locking surface to fortify the locking engagement between end cap and base section locking surfaces.

11. An implant for insertion into an intervertebral space between a first and second vertebral member, the implant comprising:
an implant body with at least one base section, the base section including
positioning teeth and a male locking surface; and
an end cap adapted for selective positioning at a selected point on the base section, the end cap comprising
an exterior contact surface that faces away from the implant body when the end cap is positioned on the base section,
a seating surface adapted to contact the base section when the end cap is positioned on the implant body,
an end cap angulation,
positioning passages extending between the exterior contact surface and the seating surface and adapted to receive positioning teeth, the positioning passages and positioning teeth complimentarily configured to facilitate positioning the end cap on the base section, and
a female locking surface complimentarily configured to lockingly engage the end cap on the base section;
wherein the implant imparts the end cap angulation to an adjacent vertebral body at the selected point when the implant is positioned in the intervertebral space.

12. The implant of claim 11, wherein the end cap is selectively positioned on the base section via rotational adjustment of the end cap about an implant longitudinal axis so that the end cap angulation coincides to the selected point.

13. The implant of claim 11, wherein
the male locking surface and female locking surface comprise a tapered configuration;
wherein the male locking surface and female locking surface have opposing and complimentary configurations that together provide a locking engagement when the end cap is positioned on the base section.

14. The implant of claim 11, wherein the end cap angulation comprises an angular value in the range of between zero degrees to fifteen degrees (0°-15°.

15. The implant of claim 11, wherein the end cap angulation comprises is an angular value selected from the group consisting of 0°, 4°, 8° and 15°.

16. The implant of claim 11, wherein the end cap further comprises an end cap height measured relative to the seating surface which enables the implant to both impart end cap height and end cap angulation to the adjacent vertebral body at the selected point.

17. The implant of claim 13, wherein the locking engagement is an interference fit, a compression fit, a friction fit or a press fit.

18. The implant of claim 11, wherein the positioning passages are spaced and located to enable complimentarily alignment with the positioning teeth to permit the end cap to be selectively positioned on the base section.

19. The implant of claim 18, wherein the positioning passages are spaced and located equidistantly about the end cap.

20. The implant of claim 11, wherein the end cap further comprises a flexible slot adapted to permit the end cap to deflect when the end cap is positioned on the base section and thereby impart a friction holding force on the complimentary male locking surface and female locking surface to fortify the locking engagement between the male locking surface and female locking surface.
